(19) United States
(12) Patent Application Publication
(10) Pub. No.: US 2013/0065303 A1
(43) Pub. Date: Mar. 14, 2013

(54) METHOD FOR INDUCING SPHEROID FORMATION OF ADIPOSE-DERIVED STEM CELLS AND TRANS-DIFFERENTIATION INTO NEURAL LINEAGE

(71) Applicants: Chia-Ching Wu, Tainan (TW); Yuan-Yu Hsueh, Tainan (TW); Sheng-Che Lin, Tainan (TW)

(72) Inventors: Chia-Ching Wu, Tainan (TW); Yuan-Yu Hsueh, Tainan (TW); Sheng-Che Lin, Tainan (TW)

(21) Appl. No.: 13/541,854

(22) Filed: Jul. 5, 2012

Related U.S. Application Data
(60) Provisional application No. 61/504,403, filed on Jul. 5, 2011.

(51) Int. Cl. C12N 5/0797

(52) U.S. Cl. 435/377

(57) ABSTRACT

The present invention relates to a basal medium for spheroid formation of adipose-derived stem cells, comprising a substrate; and a chitosan film faulted on a surface of the substrate, wherein the chitosan film comprises chitosan with 60-90% degree of deacetylation, and the chitosan film has a surface roughness defined by a height difference, measured between a highest position and a lowest position thereof, of 1-25 nm. In addition, the present invention further provides a method for inducing the spheroid trans-differentiating into neural lineages by using the basal medium of the present invention.

### CROSS REFERENCE TO RELATED APPLICATION

This application claims the benefit of filing date of U.S. Provisional Application Ser. No. 61/504,403, entitled “Spheroid formation and neural induction in human adipose-derived stem cells on chitosan coated surface” filed Jul. 5, 2011 under 35 USC §119(e)(1).

### BACKGROUND OF THE INVENTION

1. Field of the Invention

The present invention relates to a method for growing spheroid-derived adipose-derived stem cells and, more particularly, to a basal medium and a method for inducing spheroid formation and neural induction of adult adipose-derived stem cells without adding any neurotrophic factors.

2. Description of Related Art

Central nervous system (CNS) injury is an injury to the nervous tissue with long-lasting conditions that does not involve self-regeneration and recovery. Recovery for peripheral nervous system (PNS) injury, on the other hand, can occur at a rate of approximately 50-70% for patients of younger age, yet for senile patients, nerve recovery from the same injury can lower and full regeneration may not necessarily occur. While the use of nervous stem cell (NSCs) transplant for repair by means of replacing, supporting, or inducing central nerve or peripheral nerve, or the use of embryonic stem cell transplant have been common practices for neuroregeneration for severe nervous tissue injury, the use of embryonic stem cell itself is criticized for lack of capability to overcome incompatibility reaction in clinical transplant and for the ethical issues thereof. Such issues are called upon and significant even in light of embryonic stem cell's unlimited replication and differentiation ability. Similarly for the use of adult neural stem cell, even though adult neural stem cell also promises replication and differentiation ability, and has less potential facing incompatibility reaction issue, drawbacks of adult neural stem cell are that cell stock is difficult to come by in terms of quantity, its replication is limited, and it requires intervention stimulated by different neural nutrient factor in order to differentiate into different neural cells, which can significantly decrease the success rate of transplanting neural stem cell.

Several papers have investigated how to culture stem cells, these studies show that replication number, sphere formation of adult stem cell, and others are the challenges that require research progress. In the known culture conditions for stem cells, some biocompatible materials are used in a culture medium, such as chitin, hyaluronic acid and lactic-co-glycolic acid. In addition, some nutrition factors or inducing growth factors may also be added into the culture medium to induce stem cell differentiation. However, the culture process for adult stem cells is complicated and the amount of stem cells obtainable may not be enough for adult stem cell transplant to those patients in need, adding to them risk from operation difficulty and costly medical expense too great to be bearable. Hence, it is desirable to provide a low-cost culture method for adult stem cells, which can improve the culture conditions to make the adult stem cells form into spheres with differentiation capability, and induce the cells differentiating into neural lineages for transplanting to those patients with severe nerve injuries.

### SUMMARY OF THE INVENTION

An object of the present invention is to provide a basal medium for spheroid formation of adipose-derived stem cells by using a chitosan film, wherein adipose-derived stem cells self-aggregating into spheroids can be obtained by use of the basal medium of the present invention.

Another object of the present invention is to provide a method for inducing spheroids trans-differentiating into neural lineages, wherein the medium used to culture the adipose-derived stem cells can induce them to trans-differentiate into neural lineage.

To achieve the object, the present invention provides a basal medium for spheroid formation of adipose-derived stem cells, comprising: a substrate; and a chitosan film formed on a surface of the substrate. Herein, the chitosan film comprises chitosan with 60-90% degree of deacetylation (DA). Preferably, the chitosan contained in the chitosan film has 80-90% degree of deacetylation. More preferably, the chitosan contained in the chitosan film has about 85% degree of deacetylation. In addition, the chitosan film preferably has a smooth surface, wherein the chitosan film can have a surface roughness defined by a height difference, between a highest position and a lowest position thereof, of within 30 nm. Preferably, the height difference defining the surface roughness of the chitosan film, between a highest position and a lowest position, is 1-25 nm. More preferably, the height difference defining the surface roughness of the chitosan film, between a highest position and a lowest position, is 15-20 nm.

In the basal medium of the present invention, the chitosan film may be formed by coating the surface of the substrate with a 1-5% w/v chitosan solution through any coating manner. Preferably, the concentration of the chitosan solution is 1-3% w/v. More preferably, the concentration thereof is 1-2% w/v.

In addition, the basal medium of the present invention may further comprise a nutrition factor, which can be added into the chitosan film through any conventional manner. For example, the nutrition factor is added into the chitosan solution before performing the coating process of the chitosan solution, or the nutrition factor is formed directly on the surface of the chitosan film. The nutrition factor can be any factor that can induce growth or differentiation of the adipose-derived stem cells, but is not particularly limited. The nutrition factor of the present invention can be at least one selected from the group consisting of nerve growth factor (NGF), brain derived neurotrophic factor (BDNF), glial cell derived neurotrophic factor (GDNF), basic fibroblast growth factor (bEGF) and epidermal growth factor (EGF). Preferably, the nutrition factor is selected from the group consisting of nerve growth factor (NGF), brain derived neurotrophic factor (BDNF), basic fibroblast growth factor (bEGF) and epidermal growth factor (EGF). More preferably, the nutrition factor is selected from the group consisting of nerve growth factor (NGF) and brain derived neurotrophic factor (BDNF).

To achieve another object, the present invention further provides a method for inducing spheroid trans-differentiating into the neural lineage, which comprises the following steps: (A) providing the aforementioned basal medium for spheroid formation of adipose-derived stem cells of the present invention; and (B) seeding adipose-derived stem cells on the basal medium to induce a spheroid formation and a sequential neural induction of the adipose-derived stem cells. Herein, since the basal medium used in the present method is the same as that illustrated above, a detailed description of the properties thereof will not be reiterated. Since the seeding density of cells is one important factor in the spheroid formation and the neural induction of the adipose-derived stem cells, the seeding density thereof has to be 5×10³-160×10³ cells/cm² in step (B) of the method of the present invention. Preferably, the seeding density thereof is 10×10³-50×10³ cells/cm². More preferably, the seeding density thereof is 15×10³-25×10³ cells/cm².

In the aforementioned steps of the method of the present invention, the adipose-derived stem cells can be cultured on the basal medium for 96 hr to induce spheroid formation and neural induction of the adipose-derived stem cells. Preferably, the cells were cultured on the basal medium for 24-96 hr. More preferably, the cells were cultured on the basal medium for 24-72 hr.

The basal medium and the method of the present invention are mainly used to culture animal or human adipose-derived stem cells in vitro. In comparison with the conventional medium and method for culturing stem cells with neurotrophic factors, the spheroid formation and the neural induction of the adipose-derived stem cells can be achieved by only using the basal medium containing chitosan with a specific degree of deacetylation and surface roughness but without using any neurotrophic factors. By using the basal medium and the method of the present invention, the adipose-derived stem cells can be cultured to have sphere forms and lineage differentiation capabilities. Hence, when the cultured adipose-derived stem cells are induced to differentiate into neural lineages by using the basal medium and the method of the present invention, the differentiated cells can be applied to nerve injury repair.

Other objects, advantages, and novel features of the invention will become more apparent from the following detailed description when taken in conjunction with the accompanying drawings.

### BRIEF DESCRIPTION OF THE DRAWINGS

FIG. 1A shows an AFM scanning photo of the basal medium according to Embodiment 1 of the present invention;

FIG. 1B shows a quantitative curve of FIG. 1A;

FIG. 1C shows a phase image of hADSCs according to Embodiment 2 of the present invention;

FIG. 2 shows phase image of hADSCs in different seeding densities and incubation times according to Embodiment 3 of the present invention;

FIG. 3A shows the relation between the survival rates and different seeding densities of hADSCs according to Embodiment 3 of the present invention;

FIG. 3B shows the relation between the survival rates and different incubation times of hADSCs seeded with a seeding density of 2×10⁴ cells/cm² according to Embodiment 3 of the present invention;

FIG. 4A shows epi-fluorescent images of neural markers in hADSCs labeled with a fluorescence according to Embodiment 4 of the present invention;

FIG. 4B shows a result of western blotting of hADSCs according to Embodiment 4 of the present invention;

FIG. 4C shows results of epi-fluorescent images of neural markers in hADSCs labeled with a fluorescence according to Embodiment 4 of the present invention;

FIG. 4D shows a quantitative result of epi-fluorescent images of neural markers in hADSCs labeled with a fluorescence according to Embodiment 4 of the present invention;

FIG. 5A shows epi-fluorescent images of neural markers in hADSCs labeled with a fluorescence according to Embodiment 5 of the present invention; and

FIG. 5B shows gene expressions of neural markers in hADSCs according to Embodiment 5 of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

In the following embodiments, data were obtained from at least three independent experiments for each time point and each sample, with the standard deviation of the value of each data assigned as positive and negative. All the quantitative data were analyzed by independent t test. The p value less than 0.05 means significance.

### Embodiment 1

### Preparation of Basal Medium with Chitosan Film

Chitosan powder (degree of deacetylation=85%; 417963, Sigma, St. Louis, Mo.) was dissolved in IM acetic acid to obtain the concentration of 1% w/v after 24 hours (hr) agitation and then filtered twice to remove impurities. 1 ml of the obtained chitosan solution were added into each well of 12-well tissue culture plate (TCPS), and dried at 60° C. for 24 hr to obtain a chitosan film. Next, 1N of NaOH was added thereto to neutralize the coated chitosan film, and then the neutralized plate was washed with distilled water and exposed to UV light for 12 hr. After the aforementioned process, the basal medium with a chitosan film of the present embodiment was obtained.

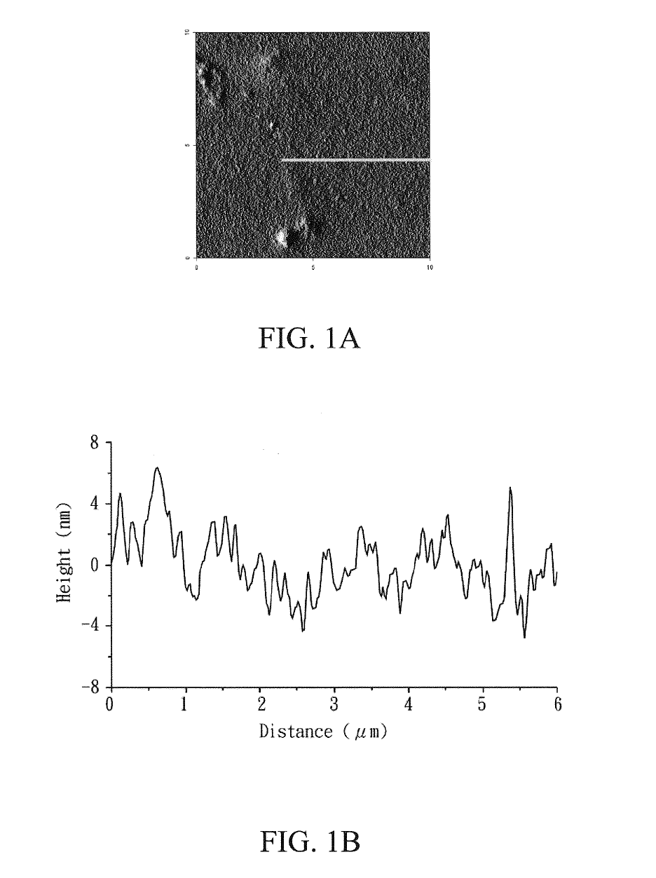

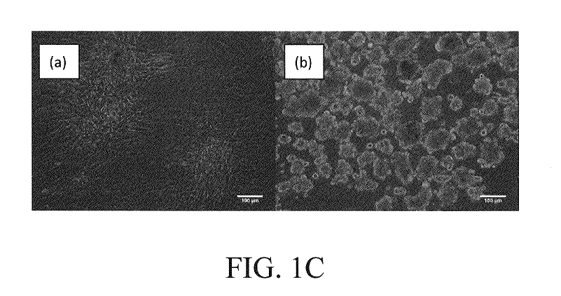

The topography and roughness of the chitosan film of the basal medium of the present embodiment were observed by scanning the surface of the chitosan film with atomic force microscopy (AFM, JPK Nanowizard II, Berlin, Germany), wherein the scanning was performed under following parameters: frequency=1 Hz, voltage=1 mV, iGain=130, and PGain=0.003. The diagram was analyzed using the JPKSPM image processing software, and the analyzed results are shown in FIG. 1A and FIG. 1B. FIG. 1A is an AFM scanning photo of the basal medium of Embodiment 1 of the present invention, and FIG. 1B is a quantitative curve of FIG. 1A. As shown in FIG. 1B, the chitosan film has a surface roughness marked by a height difference, between a highest position and a lowest position thereof, of within 20 nm, as given by the scanning distance of 6.742 μm.

### Embodiment 2

### Culture of Adipose-Derived Stem Cells into Spheres

Human adipose-derived stem cells (hADSCs) used in the present embodiment were obtained from volunteer patients of NCKU hospital. The hADSCs isolated from tissues were cultured in DNEN (Dulbecco's modified Eagle's medium, DEME, Invitrogen Inc., Carlsbad, Calif.) consisting of 10% FBS and 1% penicillin-/streptomycin at 37° C. in 5% CO₂ for the following experiments. To maintain high differentiation ability, all hADSCs used in the following embodiments were within 10 passages.

hADSCs were respectively cultured in the basal medium prepared in Embodiment 1 and a conventional TCPS plate for 48 hr. The phase images are shown in FIG. 1C. The figure (a) in FIG. 1C is the phase image of hADSCs cultured in TCPS plate, and the figure (b) in FIG. 1C is the phase image of hADSCs cultured in the basal medium of Embodiment 1. As shown in FIG. 1C, hADSCs cultured in the basal medium with the chitosan film can self-aggregate into spheres having a diameter of 100-200 μm. However, hADSCs cultured in the conventional TCPS plate cannot aggregate into spheres, and hADSCs with differentiation capabilities cannot be obtained by using the conventional TCPS plate.

### Embodiment 3

### Optimization Culture Conditions for Sphere Formation of hADSCs

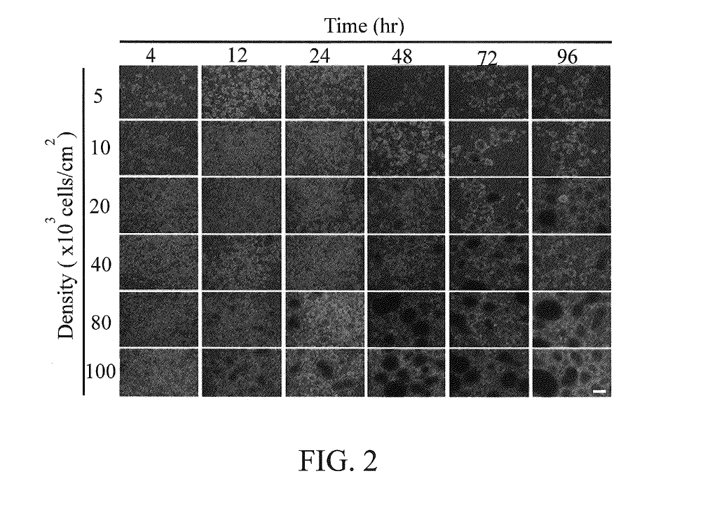

hADSCs obtained from Embodiment 2 were seeded in the basal medium prepared in Embodiment 1. In the present embodiment, different seeding densities (5×10³, 1×10⁴, 2×10⁴, 4×10⁴, 8×10⁴ and 1.6×10⁵ cells/cm²) and different culture times (4, 12, 24, 72 and 96 hr) were tested. The phase images of the cultured hADSCs are shown in FIG. 2. As shown in FIG. 2, sphere formations of hADSCs can be observed when the cells were cultured for 12 hr. As incubation time increased, the sphere size was increased and the number of spheres was decreased. In addition, as the seeding density and the incubation time of the cells increased, single cells without aggregation into spheres were subjected to anoikis induced cell apoptosis. The following Table 1 shows the relations between the distributions of sphere numbers and the populations for sphere sizes among different seeding densities. When the seeding density of hADSCs was 2×10⁴ cells/cm², a maximum distribution of sphere number of hADSCs can be obtained. In other words, a maximum number of hADSCs with differentiation capabilities can be obtained when hADSCs were seeded with 2×10⁴ cells/cm² of seeding density.

TABLE 1

| Seeding | | | | | | | |
|---|---|---|---|---|---|---|---|
| density | | 12 hr | | 48 hr | | 96 hr | |
| (x10³ | Diameter | No. | | No. | | No. | |
| cells/ | of sphere | (sphere/ | | (sphere/ | | (sphere/ | |
| cm²) | (D) (μm) | mm²) | % | mm²) | % | mm²) | % |
| 5 | 50 < D < | 24.8 ± 2.2 | 88.1 ± 2.8 | 6.61 ± 1.8 | 82.3 ± 3.5 | 4.3 ± 1.5 | 69.2 ± 11.9 |
| | 100 | | | | | | |
| | 100 < D | 3.3 ± 0.8 | 11.9 ± 2.8 | 1.2 ± 0.3 | 14.7 ± 1.8 | 1.5 ± 0.3 | 26.0 ± 9.8 |
| | < 200 | | | | | | |
| | 200 < D | 0.0 ± 0.0 | 0.0 ± 0.0 | 0.2 ± 0.1 | 3.0 ± 2.0 | 0.3 ± 0.1 | 4.3 ± 2.2 |
| 10 | 50 < D < | 35.7 ± 4.3 | 83.3 ± 6.3 | 11.7 ± 1.2 | 82.1 ± 3.3 | 3.8 ± 1.5 | 62.9 ± 11.0 |
| | 100 | | | | | | |
| | 100 < D | 6.2 ± 2.2 | 14.4 ± 4.5 | 2.2 ± 0.7 | 15.0 ± 2.8 | 1.3 ± 0.2 | 23.1 ± 1.6 |
| | < 200 | | | | | | |
| | 200 < D | 1.0 ± 0.8 | 2.3 ± 2.0 | 0.4 ± 0.1 | 2.9 ± 0.4 | 0.8 ± 0.3 | 14.0 ± 9.4 |
| 20 | 50 < D < | 9.3 ± 0.7 | 54.9 ± 0.4 | 6.3 ± 0.9 | 55.3 ± 4.0 | 3.1 ± 0.3 | 49.7 ± 9.2 |
| | 100 | | | | | | |
| | 100 < D | 6.2 ± 0.7 | 36.2 ± 1.7 | 3.9 ± 0.5 | 34.0 ± 2.8 | 2.2 ± 0.6 | 34.7 ± 7.7 |
| | < 200 | | | | | | |
| | 200 < D | 1.5 ± 0.2 | 8.9 ± 2.0 | 1.2 ± 0.2 | 10.8 ± 2.4 | 1.0 ± 0.5 | 15.6 ± 6.3 |
| 40 | 50 < D < | 10 ± 1.2 | 55.0 ± 1.2 | 4.9 ± 0.7 | 47.1 ± 6.0 | 3.7 ± 0.8 | 54.6 ± 12.3 |
| | 100 | | | | | | |
| | 100 < D | 6.3 ± 0.6 | 34.4 ± 1.6 | 3.8 ± 1.1 | 36.0 ± 2.0 | 1.6 ± 0.5 | 23.5 ± 6.5 |
| | < 200 | | | | | | |
| | 200 < D | 1.9 ± 0.4 | 10.5 ± 1.4 | 1.8 ± 0.7 | 16.9 ± 4.2 | 1.5 ± 0.4 | 21.9 ± 5.8 |
| 80 | 50 < D < | 7.3 ± 2.1 | 47.4 ± 1.9 | 5.5 ± 0.6 | 53.0 ± 6.0 | 2.3 ± 1.4 | 39.9 ± 5.5 |
| | 100 | | | | | | |
| | 100 < D | 6.5 ± 1.9 | 42.5 ± 1.9 | 3.1 ± 0.5 | 29.9 ± 4.4 | 1.8 ± 1.3 | 30.6 ± 6.9 |
| | < 200 | | | | | | |
| | 200 < D | 1.5 ± 0.2 | 10.1 ± 1.5 | 1.8 ± 0.2 | 17.1 ± 1.4 | 1.4 ± 0.2 | 29.6 ± 12.3 |
| 160 | 50 < D < | 4.8 ± 0.6 | 39.8 ± 3.8 | 3.8 ± 0.4 | 39.7 ± 2.5 | 2.6 ± 1.3 | 42.0 ± 8.8 |
| | 100 | | | | | | |
| | 100 < D | 4.5 ± 0.5 | 37.7 ± 2.8 | 3.3 ± 0.5 | 33.8 ± 2.0 | 2.1 ± 0.8 | 34.7 ± 2.8 |
| | < 200 | | | | | | |
| | 200 < D | 2.7 ± 0.7 | 22.5 ± 6.6 | 2.6 ± 0.3 | 26.5 ± 3.2 | 1.3 ± 0.3 | 23.3 ± 9.5 |

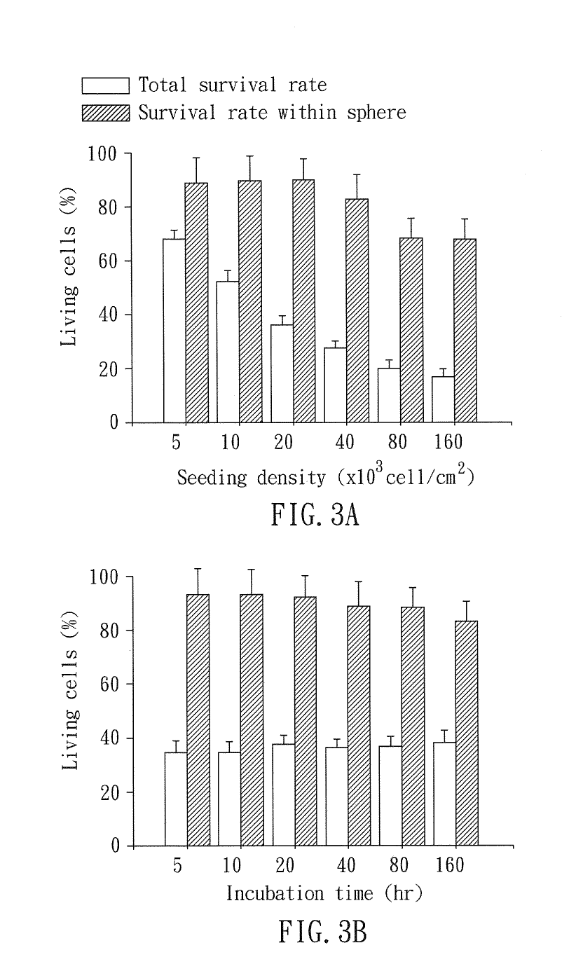

Next, samples seeded with cells having different seeding densities were subjected to Trypan Blue staining (Invitrogen) to examine the cell viability in whole populations (i.e. total survival rate) and the relative survival rate of hADSCs within spheres. To represent total survival rate, both the spheres and individual cells floating in the medium were collected, centrifuged, trypsinized into individual cells, re-suspended at the concentration of 1×10⁵ cells/ml, and then mixed with Trypan Blue for 5 min The nonviable (stained in blue color) and viable (opaque) cells were counted under a hemocytometer to determine cell survival rate. To represent the relative survival rate of hADSCs in spheres, only the hADSCs in spheres were collected, centrifuged, trypsinized into individual cells, re-suspended and then mixed with Trypan Blue. The cells were also counted to determine the relative survival rate of hADSCs within spheres. The results are shown in FIG. 3, wherein FIG. 3A shows the total survival rate and the relative survival rate of hADSCs within spheres when hADSCs were seeded with seeding densities of 5×10³, 1×10⁴, 2×10⁴, 4×10⁴, 8×10⁴ and 1.6×10⁵ cells/cm² and incubated for 48 hr. As shown in FIG. 3A, as the seeding densities of hADSCs increased, the total survival rate was decreased. However, the relative survival rate of hADSCs within spheres did not decrease. In addition, the results also show that the optimum seeding density for hADSCs is 2×10⁴ cells/cm². Furthermore, FIG. 3B shows the total survival rate and the relative survival rate of hADSCs within spheres when hADSCs were seeded with a seeding density of 2×10⁴ cells/cm² and cultured for different culture times. As shown in FIG. 3B, both the total survival rate (36%) and the relative survival rate of hADSCs within spheres (91%) do not decrease as he incubation time increases. Only slight decrease of the total survival rate and the relative survival rate of hADSCs within spheres was observed until after 72 hr.

The aforementioned results indicate that the optimal condition for the basal medium of Embodiment 1 to culture hADSCs is a seeding density of 2×10⁴ cells/cm² and an incubation time of 72 hr. When hADSCs were cultured in this condition, maximal number of hADSCs in sphere with best quality can be obtained.

### Embodiment 4

### Introduction of hADSCs Differentiating into Neural Lineage

To examine the differentiation of hADSCs into neural lineage, hADSCs were respectively seeded in the basal medium prepared in Embodiment 1 and a conventional TCPS plate with a seeding density of 2×10⁴ cells/cm², and then incubated for 72 hr. When the adipose-derived stem cells differentiate into neural lineages, nestin, NFH and GFAP are expressed on the cell surface. Hence, in the present embodiment, nestin, NFH and GFAP were used as neural precursor markers to determine whether hADSCs differentiated into neural lineages or not.

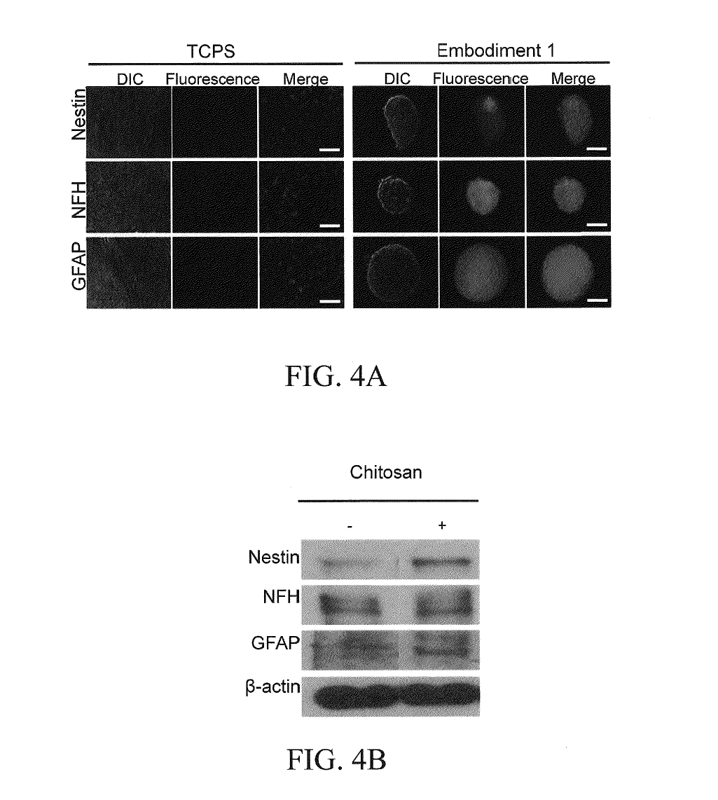

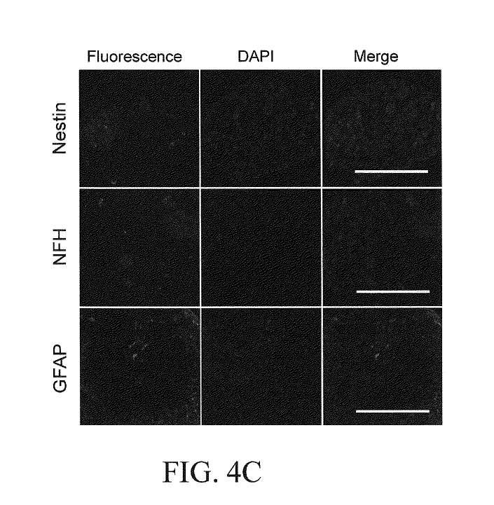

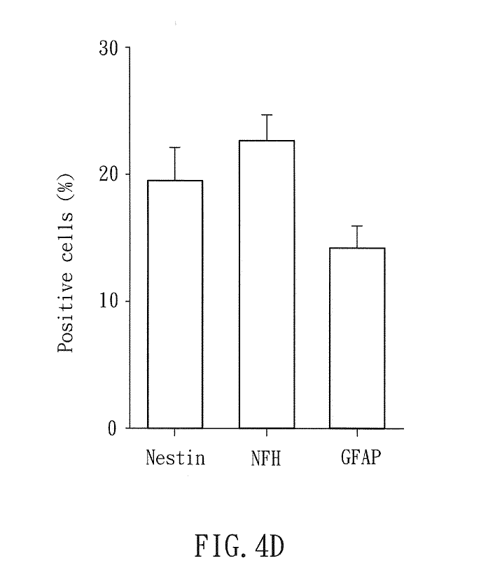

FIG. 4A shows epi-fluorescent images of hADSCs cultured in the basal medium prepared in Embodiment 1 and a conventional TCPS plate. The process to obtain the epi-fluorescent images of hADSCs is shown as follows. First, cultured hADSCs were fixed with 4% formaldehyde, permeabilized in 0.1% Triton X-100, and blocked with 1% horse serum in PBS for 30 min to prevent nonspecific antibody binding. Next, the cells were respectively incubated with primary antibodies against nestin (1:250, P48681, Millipore), NFH (1:250, P12036, Millipore) and GFAP (1:500, P14136, Millipore) at 4° C. overnight. Then, the samples were incubated with FITC or Alexa Fluor 568(1:250, Molecular Probe, Eugene, Oreg.) conjugated with secondary antibodies for 1 hr. As shown in FIG. 4A, the results indicate that nestin, NFH and GFAP were expressed on the cell surfaces of hADSCs incubated on the basal medium of Embodiment 1, but these neural precursor markers were not observed on hADSCs incubated on the conventional TCPS plate.

In addition, the protein expressions of nestin, NFH and GFAP were also examined with western blotting by using nestin (1:500, P48681, Millipore), NFH (1:100, P12036, Millipore) and GFAP (1:2000, P14136, Millipore). As shown in FIG. 4B, significant protein expressions of nestin, NFH and GFAP can be observed in hADSCs incubated on the basal medium of Embodiment 1, in comparison with those incubated on the conventional TCPS plate.

Furthermore, the nuclei of hADSCs incubated on the basal medium of Embodiment 1 were visualized by staining of DAPI (Invitrogen). Herein, nestin-, NFH- and GFAP-positive cells within the sphere were observed by using confocal microscopy, and the ratio of positive cells in different neural precursor markers was also calculated. As shown in FIG. 4C and FIG. 4D, nestin-positive hADSCs were presented on the periphery of sphere and clustered within sphere, NFH-positive hADSCs aggregated in sphere, and GFAP-positive hADSCs were presented on the periphery of sphere. In addition, the composition of nestin, NFH, and GFAP positive cells within the sphere is 19.5±2.6%, 22.6±2.0%, and 14.3±1.7%, respectively.

From the results of the present embodiment, significant expressions of nestin, NFH and GFAP were observed within the hADSCs incubated on the basal medium of Embodiment 1. These results indicate that the cultured hADSCs indeed can form in sphere and differentiate into neural lineages.

### Embodiment 5

### Passages of Adipose-Derived Stem Cells

Adipose-derived stem cells (hADSCs) derived from patients in Embodiment 2 were subcultured for three times, and the expressions of nestin, NFH and GFAP were examined by the same method used in Embodiment 4. In the present embodiment, the primary spheres of hADSCs formed after incubation for 48 hr were trypsinized, dispersed into individual cells, and then replated on another new basal medium prepared in Embodiment 1. The aforementioned process was performed two times to obtain secondary spheres and tertiary spheres.

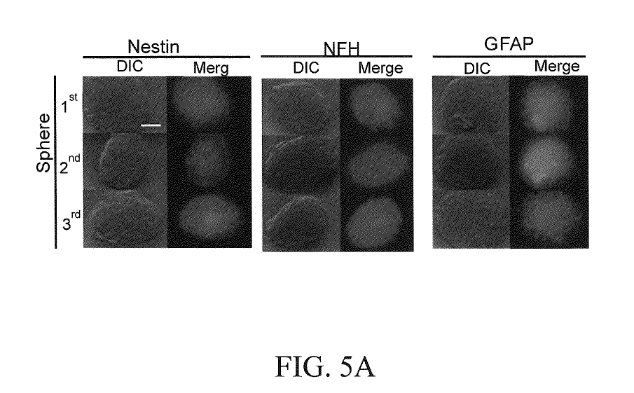

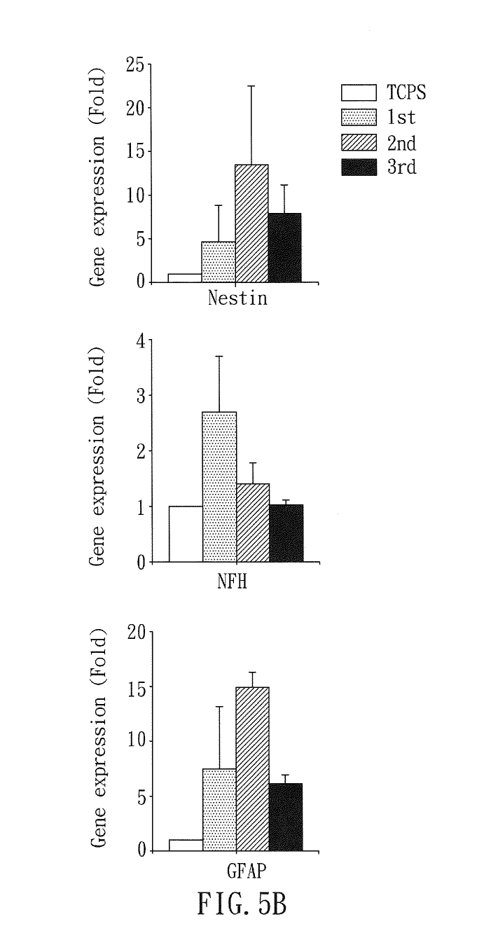

The fluorescent results of the expressions of nestin, NFH and GFAP in hADSCs are shown in FIG. 5A. As shown in FIG. 5A, the expressions of neural precursor markers in protein levels were increased in primary spheres, further enhanced in secondary spheres, and minor reduced in tertiary spheres.

In addition, a quantitative real time polymerase chain reaction (real time PCR) was also used to detect the gene expressions of neural precursor markers such as nestin, NFH and GFAP in hADSCs, which were cultured on both the basal medium of Embodiment 1 and the conventional TCPS plate. Herein, the mRNA of hADSCs were isolated using RNeasy mini kit (QIAGEN, 74106, Germany), and a reverse transcription-polymerase chain reaction (RT-PCR) was carried out by ReverTra Ace (TOYOBO, FSK101, JAPAN) to reverse-transcript mRNA into cDNA. The sequence of the individual primer was shown in the following Table 2. Finally, quantitation of cDNA of neural precursor markers was performed with the LightCycler TaqMan Master Kit (Roche Diagnostics, 04-535-286-001, Germany), using a LightCycler Instrument (LightCycler System, Roche Diagnostics, Germany). As shown in FIG. 5B, less gene expressions of neural precursor markers were observed in hADSCs cultured on the conventional TCPS plate, but significant gene expressions of neural precursor markers were observed in hADSCs cultured on the basal medium of Embodiment 1. These results indicate that adipose-derived stem cells can form in sphere when cultured on basal medium with a chitosan film, and furthermore the cultured cells can differentiate into neural lineage without adding any inducing nutrition factors.

TABLE 2

| Gene | Forward sequence | Reverse sequence |
|---|---|---|
| Sequence | (5′→6′) | (5′→3′) |
| β-Actin | ccaaccgcgagaagatga | ccagaggcgtacagggatag |
| Nestin | tgcgggctactgaaaagttc | tgtaggccctgtttctcctg |
| NFH | ccgacattgcctcctacc | ggccatctcccacttggt |
| GFAP | ccaacctgcagattcgaga | tcttgaggtggccttctgac |

According to the results obtained from Embodiments 1-5, stem cells cultured on the basal medium with a chitosan film containing nutrition factors of the present invention can form in spheres and differentiate into neural lineages (data not shown). In addition, adipose-derived stem cells cultured on the basal medium with a chitosan film only containing chitosan of the present invention can also aggregate into spheres and have differentiation capabilities, as shown in FIG. 1. Furthermore, the present invention also provides an optimal condition to obtain maximum amount of sphere cell clusters and high survival rate, wherein the optimal condition is a seeding density of 2×10⁴ cells/cm² and an incubation time of 72 hr, as shown in FIG. 2 and FIG. 3. In addition, the inventors also confirmed that the adipose-derived stem cells cultured on the basal medium of the present invention indeed have neural differentiation capabilities. According to the results of fluorescent staining, western blotting and real-time PCR, those results indicate that the adipose-derived stem cells cultured on the basal medium without adding any nutrition factors of the present invention indeed have neural differentiation capabilities, as shown in FIG. 4 and FIG. 5. Hence, the basal medium and the method of the present invention can be used to easily culture adipose-derived stem cells into sphere clusters with differentiation capabilities, and the obtained stem cells with these capabilities can be used as new therapeutic means for treating patients with neural diseases.

Although the present invention has been explained in relation to its preferred embodiment, it is to be understood that many other possible modifications and variations can be made without departing from the spirit and scope of the invention as hereinafter claimed.

1. A basal medium for spheroid formation of adipose-derived stem cells, comprising:
a substrate; and
a chitosan film formed on a surface of the substrate, wherein the chitosan film comprises chitosan with 60-90% degree of deacetylation, and the chitosan film has a surface roughness defined by a height difference, measured between a highest position and a lowest position thereof, of 1-25 nm.

2. The basal medium as claimed in claim 1, wherein the chitosan contained in the chitosan film has 80-90% degree of deacetylation.

3. The basal medium as claimed in claim 1, wherein the chitosan film is formed by coating the surface of the substrate with a 1-5% w/v chitosan solution.

4. The basal medium as claimed in claim 1, wherein the chitosan film further comprises a nutrition factor selected from the group consisting of nerve growth factor (NGF), brain derived neurotrophic factor (BDNF), glial cell derived neurotrophic factor (GDNF), basic fibroblast growth factor (bEGF) and epidermal growth factor (EGF).

5. The basal medium as claimed in claim 1, wherein the chitosan film has the surface roughness defined by a height difference, measured between a highest position and a lowest position thereof, of 15-20 nm.

6. A method for inducing spheroid trans-differentiating into neural lineages, comprising:
(A) providing a basal medium for spheroid formation of adipose-derived stem cells, which comprises:
a substrate; and
a chitosan film formed on a surface of the substrate, wherein the chitosan film comprises chitosan with 60-90% degree of deacetylation, and the chitosan film has a surface roughness defined by a height difference, measured between a highest position and a lowest position thereof, of 1-25 nm; and
(B) seeding adipose-derived stem cells with a seeding density of 5×10³-160×10³ cells/cm² on the basal medium to induce a spheroid formation and a neural induction of the adipose-derived stem cells.

7. The method as claimed in claim 6, wherein the adipose-derived stem cells are cultured on the basal medium for 24-96 hr to induce the spheroid formation and the neural induction of the adipose-derived stem cells in the step (B).

8. The method as claimed in claim 6, wherein the adipose-derived stem cells are cultured on the basal medium for 24-72 hr to induce the spheroid formation and the neural induction of the adipose-derived stem cells in the step (B).

9. The method as claimed in claim 6, wherein the adipose-derived stem cells are seeded on the basal medium with a seeding density of 10×10³-50×10³ cells/cm².

10. The method as claimed in claim 6, wherein the chitosan contained in the chitosan film has 80-90% degree of deacetylation.

11. The method as claimed in claim 6, wherein the chitosan film is formed by coating the surface of the substrate with a 1-5% w/v chitosan solution.

12. The method as claimed in claim 6, wherein the chitosan film further comprises a nutrition factor selected from the group consisting of nerve growth factor (NGF), brain derived neurotrophic factor (BDNF) and glial cell derived neurotrophic factor (GDNF), basic fibroblast growth factor (bEGF) and epidermal growth factor (EGF).

13. The method as claimed in claim 6, wherein the chitosan film has the surface roughness defined by a height difference, measured between a highest position and a lowest position thereof, of 15-20 nm.
